(19) United States
(12) Patent Application Publication
(10) Pub. No.: US 2012/0199664 A1
(43) Pub. Date: Aug. 9, 2012

(54) Deer attracting device and system

(76) Inventor: Ryan Harold Eklund, Neeses, SC (US)

(21) Appl. No.: 12/931,637

(22) Filed: Feb. 7, 2011

(51) Int. Cl. A61L 9/04

(52) U.S. Cl. 239/34

(57) ABSTRACT

Straightline is a syringe with a plunger attached to the end of flexible tubing that varies in length. The flexible tubing has holes approximately every two feet. The syringe is used to pour an attractant in which then flows through the tubing by gravity or can be accelerated by using the plunger. Straightline gives the hunter the advantage by allowing the hunter to lay the tubing out days in advance. The flexible tubing varies in length and can be laid out in bedding areas, along trails, and can be added to for extra coverage. This can help prevent leaving human scent prior to the hunt.

### BACKGROUND

This version of the invention is concerned with the field of devices to attract deer and other game animals during hunting. More specifically, this version of the invention is concerned with a device that is located upon a ground or other supporting surface and is comprised of a canister containing a quantity of liquid deer scent attractant therein and a flexible hose attached to one end of the canister, the canister dispensing liquid deer scent attractant into the flexible hose and through slits along the surface of the hose for attracting deer and other game animals to the proximity of the hunter.

### PRIOR ART

During the hunting of game animals, such as deer, various methods and devices are employed to find and attract said animals and to prevent the hunter from being detected by said animals during the hunting of said animal. Attraction devices include whistles, horns, and other apparatuses, which, for example, mimic the calls and bellows of a game animal during mating season. Other devices consist of life-like decoys ranging from a goose to a doe, which are strategically located to attract another similar type of animal. In some cases, scent attractants are spread upon a ground surface or other natural features so that pheromones or other sexually-attracting odors and smells will drift and spread over an area in which game animals are thought to frequent so as to draw said game animals to the area over which the scent attractants are dispensed. Various devices and methods have been used to dispense such scent attractants, including, but not limited to absorbent pads that are located upon a ground surface or attached to a natural feature, such as a tree branch, or an article of clothing, such as beneath the shoe of a hunter. Other devices, such as mechanical systems, are more elaborate in design and construction, generally consisting of a reservoir for storage of a quantity of liquid scent attractant, delivery means for transferring liquid scent attractant from the reservoir to the immediate atmosphere, and actuating means for determining the time to dispense liquid scent attractant, comprising either a timer, motion sensor, or the like.

The aforementioned devices and methods of attracting game animals perform their desired function with varying degrees of success. Absorbent pads attached to a natural feature, such as a tree branch, generally have a limited range over which the scent attractant is dispensed as such pads are stationary. The absorbent pads and other devices that are attached to a hunter are more effective in dispensing scent attractant over a wider geographical area as the scent attractant is dispensed to the area in which the hunter is stalking the game animal. However, many of these devices are elaborate and cumbersome to use, generally interfering with the ability of the hunter to engage in the sort of physical activity necessary to stalk and hide from game animals. The mechanical devices that dispense scent attractant suffer from several disadvantages. They are generally the most expensive to purchase and maintain, and they are subject to mechanical failure in the field, thus jeopardizing their ability to attract game animals. What is needed then to overcome the aforementioned disadvantages of existing devices and methods for attracting game animals is the provision of a device that is relatively inexpensive to purchase and maintain, simple to use, and able to hold a quantity of liquid deer scent attractant sufficient to dispense scent attractant over a geographical area and for a duration needed to attract deer and other game animals for a successful hunting outing.

### DISCUSSION OF THE PRIOR ART

Numerous designs for devices and methods for attracting game animals through dispensation of animal scent attractant have been provided in the prior art. Even though these designs may be suitable for the specific individual purposes to which they address, they would not be suitable for the purposes of the present version of the invention as such designs are limited by the disadvantages and drawbacks recited earlier in this disclosure. These designs are exemplified by the following patents:
U.S. Pat. No. 4,735,010, Scent Dispenser For Attachment Under A Shoe, issued to Grinarmi on 5 Apr. 1988;
U.S. Pat. No. 5,555,665, Scent-releasing Pole For Attracting Deer, issued to Fore on 17 Sep. 1996;
U.S. Pat. No. 5,971,208, Smart Scent Dispenser, issued to Kennedy on 26 Oct. 1999;
U.S. Pat. No. 5,987,800, Scented Trail Making Device, issued to Regan on 23 Nov. 1999;
U.S. Pat. No. 6,966,500, Remote Controlled Scent Making Device, issued to Kelley on 22 Nov. 2005; and
U.S. Pat. No. 7,108,199, Device For Dispensing Liquid Scent, issued to Brown on 19 Sep. 2006.

As such, it may be appreciated that there its a continuing need for a new and improved device that dispenses animal scent attractant in order to attract deer and other game animals to the vicinity of a hunter. The deer attracting device and system that is the subject of the instant invention employs a canister with a quantity of liquid deer scent attractant situated therein and a flexible hose attached to one end of the canister. The canister consists of a hollow cylindrical member defined by first and second opposed end walls and a pump handle that is disposed within an aperture of the first end wall. A series of slits is located sequentially along the surface of the flexible hose. When the pump handle is actuated, liquid deer scent attractant contained in the canister is forced into the interior of the flexible hose and through the slits of the hose upon a ground surface or other surface upon which the device is located.

The device is relatively inexpensive to purchase and maintain. Furthermore, the device does not interfere with the physical motion of the hunter while said hunter is stalking or hiding from a game animal, said device delivering scent attractant in quantities sufficient to attract a deer of other game animal. In these respects, the present version of the invention substantially departs from the conventional concepts and designs of the prior art, and in so doing provides an apparatus that substantially fulfills this need. Additionally, the prior patents and commercial techniques do not suggest the present inventive combination of component elements arranged and configured as disclosed herein.

The present invention achieves its intended purposes, objects, and advantages through a new, useful and unobvious combination of method steps and component elements, with the use of a minimum number of functioning parts, at a reasonable cost to manufacture, and by employing only readily available materials.

### SUMMARY

The present version of the invention, which will be described in greater detail hereinafter, relates to the field of devices to attract deer and other game animals during hunting. More specifically, this version of the invention is concerned with a device that is located upon a ground or other supporting surface, such as a tree, and dispenses liquid deer scent attractant onto these surfaces for attracting deer and other game animals to the proximity of the hunter. My version of the invention overcomes all of the shortcomings listed previously, in addition to novel aspects that will be described in detail hereinafter.

Described briefly, according to a typical embodiment, the device is comprised of a canister and a flexible hose attached to one end of the canister. The canister consists of a hollow cylindrical member defined by first and second opposed end walls. A pump handle is disposed within an aperture of the first end wall and a collar is attached to the second end wall over an aperture of the second end wall. A flexible hose is attached to the collar so that it is disposed within the aperture of the second end wall. A series of slits is located at regular intervals on the surface of the flexible hose.

To use the device, a quantity of liquid deer scent attractant, such as doe urine, is deposited into the hollow cylindrical member of the canister. When the pump handle is actuated, liquid deer scent attractant contained in the hollow cylindrical member canister is forced through the aperture of the second end wall of the hollow cylindrical member into the interior of the flexible hose and through the slits of the hose upon a ground surface or other surface upon which the device is located. The flexible hose through the slits thereof dispenses the liquid deer scent attractant onto the surface upon which the device is situated. Depending upon the topography of the supporting surface, the flexible hose may be bent, flexed, coiled, or otherwise contorted into various shapes and configurations. As such, the flexible hose makes optimum contact with the supporting surface or other natural features to deposit liquid deer scent attractant thereon. The ability of the device to dispense liquid deer scent attractant can be enhanced by using flexible hoses of various lengths or configurations. Over time, the liquid deer scent attractant will attract one or more deer or other game animals to the area in which the hunter has traversed or is hiding.

My invention, therefore, resides not in any one of these features per se, but rather in the particular combination of all of them herein disclosed. It is distinguished from the prior art in this particular combination of all of its structures for the functions specified.

In order that the detailed description of the invention may be better understood and that the present contribution to the art can be more fully appreciated, additional features of the invention will be described hereinafter. It should be appreciated by those skilled in the art that the conception and the disclosed specific methods and structures may be readily utilized as a basis for modifying or designing other structures for carrying out the same purposes of the present invention. It should be realized by those skilled in the art that such equivalent methods and structures do not depart from the spirit and scope of the invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application nor is it intended to be limiting as to the scope of the invention in any way.

Accordingly, it is an object of my version of the invention to provide a low-cost, easy-to-manufacture, and easy-to-market deer attracting device and system.

A further object of my version of the invention is to provide an easy-to-use and versatile deer attracting device and system.

A significant object of the invention is to provide a deer attracting device and system for dispensing liquid deer scent attractant upon a supporting surface, said device comprised of a canister and a flexible hose, said hose attached to one end of the canister and dispensing liquid deer scent attractant through a series of slits located on the surface of the hose.

A final but very significant object of the invention is to provide a deer attracting device and system to attract deer and other game animals to close proximity of the geographical area in which the user is hunting.

For a better understanding of the invention, its operating advantages and the specific objects attained by its uses, reference should be made to the accompanying drawings and descriptive matter in which there is illustrated a preferred embodiment of the invention. The foregoing has outlined some of the more pertinent objects of the invention. These objects should be construed to be merely illustrative of some of the more prominent features and applications of the present invention. Many other beneficial results can be attained by applying the disclosed invention in a different manner or by modifying the invention within the scope of the disclosure. Accordingly, other objects and a fuller understanding of the invention may be had by referring to the summary of the invention and the detailed description of the preferred embodiment in addition to the scope of the invention illustrated by the accompanying drawings.

### BRIEF DESCRIPTION OF THE DRAWINGS

The foregoing and other objects, features and advantages of the invention will become more fully understood from the following description of the preferred embodiment of the invention as illustrated in the accompanying drawings in which like reference characters refer to the same parts throughout different views. The drawings are not necessarily to scale, emphasis instead being placed upon illustrating the principles of the invention.

FIG. 1 is a first perspective view of the deer attracting device and system in accordance with the present version of the invention.

FIG. 2 is a second perspective view of the deer attracting device and system in accordance with the present version of the invention.

### DRAWING REFERENCE NUMERALS

10 Deer Attracting Device and System
12 Canister
14 Cylindrical Member
16 First End Wall
18 Second End Wall
20 Pump Handle
22 Shaft
24 Ring
26 Aperture
28 Collar
30 Tube
32 First End
34 Second End
36 Slit
38 Aperture

### DESCRIPTION OF THE PREFERRED EMBODIMENT

### Description

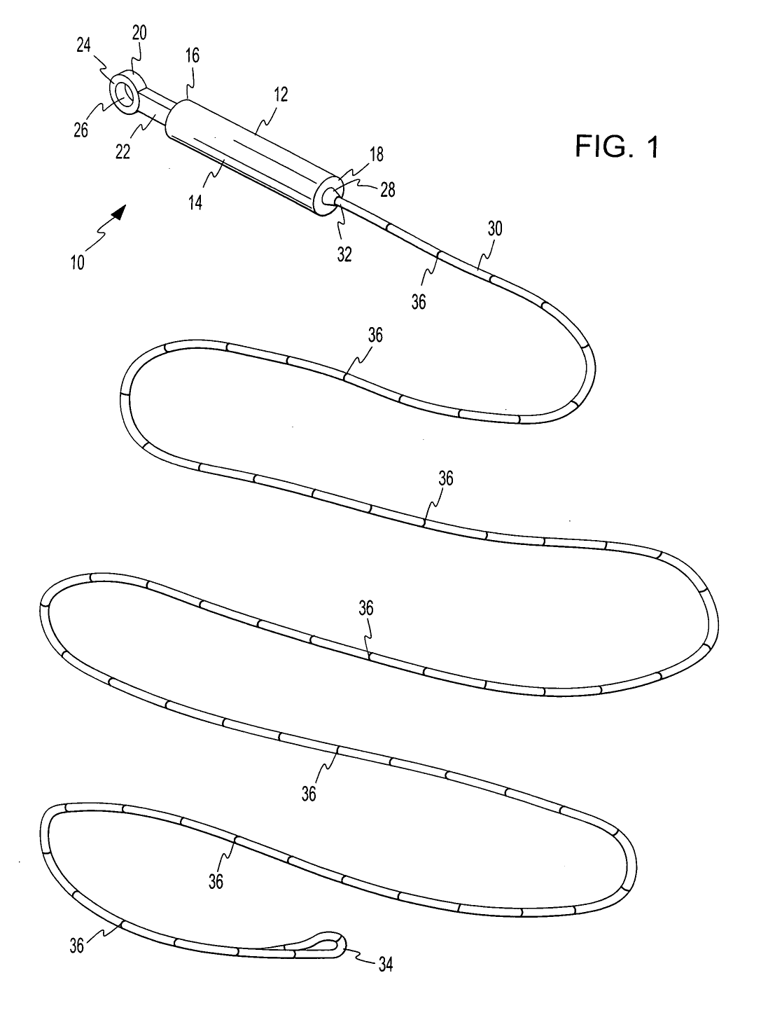

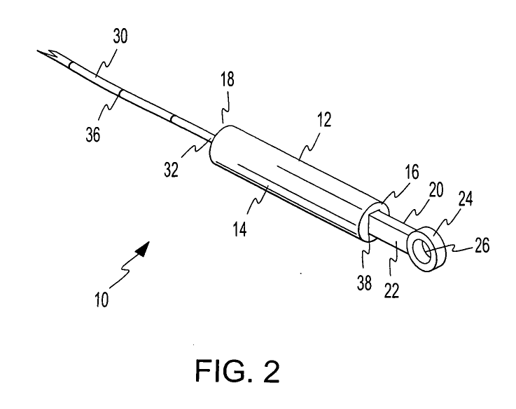

Referring now to the drawings and, in particular, to FIG. 1-FIG. 2 wherein there are illustrated a typical embodiment of the deer attracting device and system 10. The present version of the invention 10 is comprised of a canister 12 into which a quantity of liquid deer scent attractant is situated. The canister 12 is comprised of a hollow cylindrical member 14 that is defined by a first end wall 16 and a second opposed end wall 18.

A pump handle 20 is slidingly engaged within the first end wall 16 of the cylindrical member 14. The pump handle 20 is comprised of a rectangular shaft 22 and a ring 24 with central aperture 26.

A collar 28 is attached medially on the second end wall 18 of the hollow cylindrical member 14 over an aperture (not shown) of the second end wall 18. The aperture communicates with the interior of the hollow cylindrical member 14.

A hollow, flexible tube 30 having a first end 32 and a second opposed end 34 is attached to the collar 28 at the first end thereof 32. The first end 32 of the flexible hose 30 is inserted through the collar 28 and into the aperture of the second end wall 18 of the hollow cylindrical member 14 so the first end 32 of the flexible hose 30 communicates with the interior of the hollow cylindrical member 14. A series of slits 36 is located at regular intervals along the surface of the flexible hose 30. The slits 36 communicate with the interior of the flexible hose 30.

Referring now to FIG. 2, the rectangular shaft 22 of the pump handle 20 is disposed in sliding engagement within a corresponding aperture 38 located within the first end wall 16 of the hollow cylindrical member 14.

To use the device 10, a quantity of liquid deer scent attractant, such as doe urine, is deposited into the hollow cylindrical member 14 of the canister 12 through the aperture 38 located within the first end wall 16 of the hollow cylindrical member 14. When the pump handle 20 is actuated, liquid deer scent attractant contained in the hollow cylindrical member 14 of the canister 12 is forced through the aperture of the second end wall 18 of the hollow cylindrical member 14 into the interior of the flexible hose 30 and through the slits 36 of the hose 30 upon a ground surface or other surface upon which the device 10 is located. The flexible hose 30 through the slits 36 thereof dispenses the liquid deer scent attractant onto the surface upon which the device 10 is situated. Depending upon the topography of the supporting surface, the flexible hose 30 may be bent, flexed, coiled, or otherwise contorted into various shapes and configurations. As such, the flexible hose 30 makes optimum contact with the supporting surface or other natural features to deposit liquid deer scent attractant thereon. The ability of the device 10 to dispense liquid deer scent attractant can be enhanced by using flexible hoses of various lengths or configurations. Over time, the liquid deer scent attractant will attract one or more deer or other game animals to the area in which the hunter has traversed or is hiding.

While this version of the invention has been illustrated and described in detail in the drawings and foregoing description, the same is to be considered as illustrative and not restrictive in character, it being understood that only the preferred embodiment has been shown and described and that all changes and modifications that come within the spirit of the version of the invention are desired to be protected. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

### CONCLUSION AND SCOPE OF INVENTION

From the foregoing, it will be understood by persons skilled in the art that an improved deer attracting device and system has been provided. The invention is relatively simple and easy to manufacture, yet affords a variety of uses. While my description contains many specificities, these should not be construed as limitations on the scope of the version of the invention, but rather as an exemplification of the preferred embodiment thereof. The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be resorted to, falling within the scope of the invention. Although this invention has been described in its preferred form with a certain degree of particularity, it is understood that the present disclosure of the preferred form has been made only by way of example and numerous changes in the details of construction and combination and arrangement of parts may be resorted to without departing from the spirit and scope of the invention.

1. Straightline as a whole product is a scent dispersal system that allows the hunter the advantage. The system is easy to use, can be laid out days in advance and has unlimited coverage by using attachments to add extra line. This gives the hunter the advantage by allowing him/her to lay out the tubing days in advance, and avoiding leaving the human scent prior to the hunt.
